(19) United States
(12) Patent Application Publication
(10) Pub. No.: US 2006/0293530 A1
(43) Pub. Date: Dec. 28, 2006

(54) Process for the manufacture of citalopram hydrobromide

(75) Inventors: Siddiqui Mohammed Jaweed Mukarram, Aurangabad (IN); Bhargav Krishnaji Upadhye, Aurangabad (IN); Krishna Gopalji Mishra, Aurangabad (IN); Mohammed Ismail Farooqui, Aurangabad (IN)

Correspondence Address: IPLM GROUP, P.A., POST OFFICE BOX 18455, MINNEAPOLIS, MN, 55418, US

(73) Assignee: Wockhardt Ltd., Maharashtra (IN)

(21) Appl. No.: 11/414,135

(22) Filed: Apr. 28, 2006

(30) Foreign Application Priority Data
Oct. 28, 2003 (WO) PCT/IB03/04757

(51) Int. Cl. C07D 307/02

(52) U.S. Cl. 549/467

(57) ABSTRACT

The present invention describes an improved process for the preparation of pure 1-(4′-Fluorophenyl)-1-(3-dimethylaminopropyl)-5-phthalanecarbonitrile and its bromide salt (citalopram hydrobromide), which is a well known antidepressant. Another aspect of the invention is isolation of crystalline (4-Bromo-2-hydroxymethyl)phenyl-(4-fluorophenyl)-3-(dimethylaminopropyl)methanol (Bromodiol) and conversion of desmethylcitalopram which is formed during the cyanide exchange reaction, to citalopram by heating with a mixture of formaldehyde and formic acid in chloroform. The resulting citalopram is purified using conventionally extraction methodology.

### CROSS-REFERENCE TO RELATED APPLICATION

This application claims priority from international patent application Serial No. PCT/IB2003/004757, filed Oct. 28, 2003, and published in English on May 12, 2005 as International Publication No. WO 2005/042473 A1, which is incorporated herein by reference.

### FIELD OF THE INVENTION

The present invention relates to an improved process for the preparation of extremely pure 1-(4′-fluorophenyl)-1-(3-dimethylaminopropyl)-5-phthalanecarbonitrile and its bromide salt (citalopram hydrobromide), which is a well known antidepressant. Other aspects of the invention are isolation of crystalline (4-bromo-2-hydroxymethyl)phenyl-(4-fluorophenyl)-3-(dimethylaminopropyl)methanol (bromodiol) and conversion of desmethylcitalopram to citalopram generated in trace during the reaction by treatment with formaldehyde and formic acid in chloroform. The resulting citalopram product is optionally further worked up, purified and isolated in the form of a base or a pharmaceutically acceptable salts.

### BACKGROUND OF THE INVENTION

Citalopram is a selective centrally acting serotonin (5-hydroxytryptamine; 5HT) reuptake inhibitor having antidepressant activity. The activity of citalopram is described in J. Hyttel, Prog. Neuro-Psychopharmacol. & Biol. Phychiat, 1982, 6, 277-295. Its effectiveness in the treatment of dementia and cardiovascular disorder has been disclosed in EP-A 474 580. The structure of citalopram is shown in Formula (I): 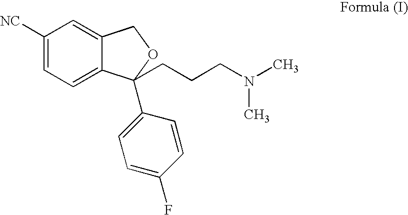
Citalopram was first discussed in DE 2,657,013, corresponding to U.S. Pat. No. 4,136,193. So far, several different processes for the preparation and purification of this active drug have been reported.

U.S. Pat. No. 4,136,193 discloses the preparation of citalopram from 5-bromophthalide using double Grignard reactions, namely with 4-fluorobromobenzene and N,N-dimethyl-aminopropyl chloride. The bromo function of 1-(4′-fluorophenyl)-1-(3-dimethylamino-propyl)-5-bromophthalan thus obtained is substituted by cyano group using copper cyanide in a suitable solvent to get the citalopram base. A small amount (1-2%, even up to 10% in some cases) of desmethylcitalopram is also found in this method which is formed during the high temperature substitution reaction.

International patents WO 2000/011926 and WO/2000 013648 disclose the use of transition metals like nickel or palladium as a catalyst for the substitution of halide group by a cyano anion, such as from KCN, NaCN or (R′₄N)CN, where R₁₄ indicates four groups which may be same or different and are selected from hydrogen and straight chain or branched C₁₋₆ alkane. Suitable leaving groups can include halides such as bromo, iodo, and leaving groups such as CF₃—(CF₂)ₙ—SO₂— wherein n is an integer from the range of 0 to 8, and the like. A preferable leaving group is CF₃—SO₂—.

On the other hand, U.S. Pat. No. 4,650,884 discloses the use of 5-cyanophthalide as the starting material for the preparation of citalopram. In the disclosed process the ring closure of the dihydroxy compound of formula 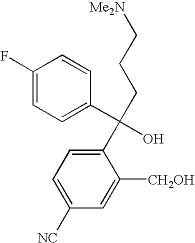
is achieved by dehydration with strong sulfuric acid at 80° C. The dihydroxy compound is prepared from 5-cyanophthalide by two consecutive Grignard reactions. A combination of various solvents is also discussed in this patent for the re-crystallization to obtain the pure citalopram.

Conversion of various functional groups such as hydroxyl, aldehyde, hydroxymethyl, nitromethyl, carboxy or methyl, etc. into a cyano functional group for the preparation of citalopram is disclosed in patents such as WO 2001/068632; WO 2001/066536; WO 1999/30548; EP 1,125,907; US 2001/056194, JP 2001/106681 and JP 2001/114773.

Apart from these, U.S. Pat. No. 6,579,993 discloses a different production method of citalopram comprising the reaction of a compound of formula 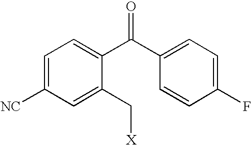
wherein X is a halogen, with organometallic dimethylaminopropyl halide.

Impurity profile of citalopram is discussed in WO 2001/47877, where a thin film distillation process is described for purification. It is well known that synthesis of citalopram in desired quality is very difficult. The manufacturing processes of citalopram described in the U.S. Pat. No. 4,136,193; WO 2000/11926, WO 2000/13648 and DE 2,657,013 comprise the exchange of 5-halogen with cyano group. It has been found that, along with citalopram, an unacceptable amount of desmethylcitalopram is also formed during the substitution of the halogen group. The removal of desmethylcitalopram is very difficult by usual work-up procedure, which leads to extensive and expensive purification processes. The chemical structure of citalopram and desmethylcitalopram is shown below:

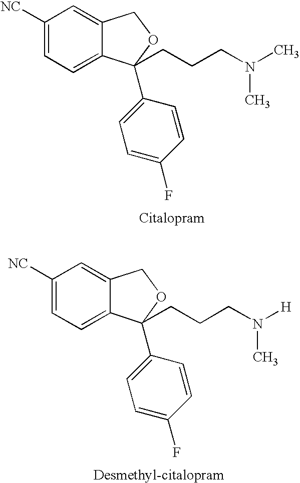

Similarly, WO 2001/045483 discloses a different purification method of citalopram. The purification method disclosed in this patent application removes desmethyl-citalopram formed during the cyanide exchange reaction. The crude citalopram obtained in this process after usual purification is subjected to treatment with an amide or an amide-like group forming agent from the agents of formulae (a), (b) or (c): 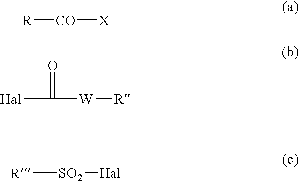
where X is halogen or a group —O—CO—R′, Hal is halogen, Y is O or S, W is O, N, or S and R′, R″ and R′″ are each selected from the group consisting of hydrogen, alkyl and optionally substituted aryl or aralkyl.

Thus, it is important to remove impurities formed during the cyanide exchange reaction in order to obtain a commercially attractive citalopram.

It is therefore an object of the present invention to provide an economical and industrially advantageous manufacture method of citalopram, which affords production of extremely pure citalopram in high yield.

### SUMMARY OF THE INVENTION

According to the present invention, a process is provided for the manufacture of highly pure 1-(4′-fluorophenyl)-1-(3-dimethylaminopropyl)-5-phthalanecarbonitrile and its bromide salt (citalaopram hydrobromide).

In a first embodiment, crystalline (4-bromo-2-(hydroxymethyl)phenyl)-(4′-fluoro-phenyl)-3-dimethylaminopropyl)methanol (bromodiol) 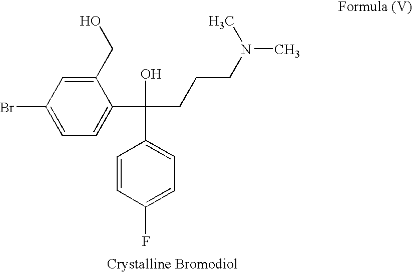
is isolated. The bromodiol is synthesized from 5-bromophthalide by two successive Grignard reactions, namely with 4-fluorobromobenzene and N,N-dimethylaminopropyl chloride.

In a second embodiment, the unwanted desmethylcitalopram formed during the cyanide exchange reaction is reconverted to citalopram by refluxing the crude citalopram with formaldehyde and formic acid in chloroform for 8 hours. The process is outlined below:

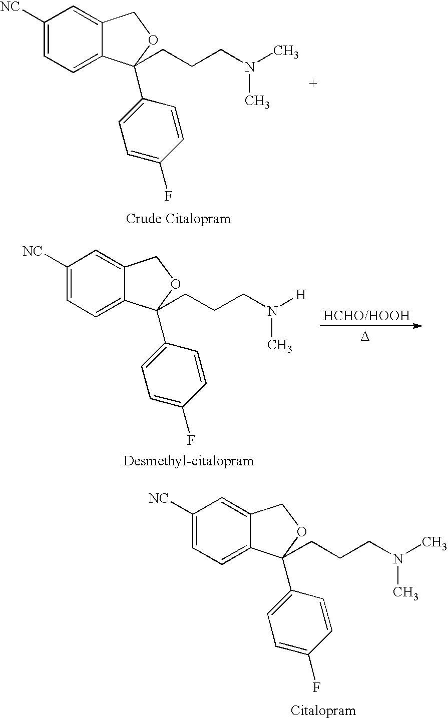

The crude citolaopram thus obtained is worked up and distilled under vacuum to get a thick oily residue. HPLC purity of the obtained citalopram is found in the range of 90-94%. Citalopram is conventionally converted to a salt such as, for example, citalopram hydrobromide using 48% hydrobromic acid in isopropyl alcohol followed by recrystallization in aqueous isopropanol. HPLC analysis showed 99.60% purity of the crystalline citalopram hydrobromide obtained using the present invention.

### DETAILED DESCRIPTION OF THE INVENTION

Citalopram is an important and active anti-depressant therapeutic agent. Accordingly, a process for the large scale manufacture of very high purity product and having control over impurities and byproducts prepared in the process is desirable. This present invention provides a manufacturing process, incorporating a step for re-conversion of desmethylcitalopram (an undesired product produced during the manufacture of citalopram) into citalopram by treatment with formic acid and formaldehyde.

The present invention is directed towards a process for manufacturing 1-(4′-fluorophenyl)-1-(3-dimethylaminopropyl)-5-phthalanecarbonitrile and its bromide salt (citalaopram hydrobromide) of Formula (I):

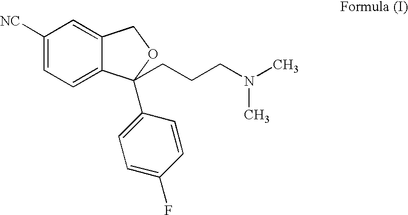

According to one embodiment, the present invention, 1-(4′-fluorophenyl)-1-(3-dimethyl-aminopropyl)-5-phthalanecarbonitrile, is prepared according to the following synthetic reaction scheme:

### (a) Reaction of 5-Bromophthalide with Magnesium halides of N,N-Dimethylaminopropyl chloride and 4-Fluorobromobenzene (Double Grignard Reaction)

5-Bromophthalide (Formula II) is reacted with magnesium halides of 4-fluorobromo benzene (Formula III) and N,N-dimethylaminopropyl chloride (Formula IV) 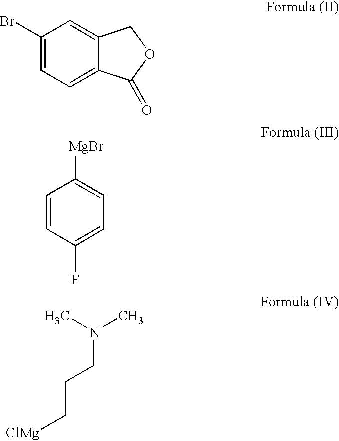
in tetrahydrofuran to form (4-bromo-2-hydroxymethyl)phenyl-(4-fluorophenyl)-3-(dimethylaminopropyl)methanol, (bromodiol, Formula V)

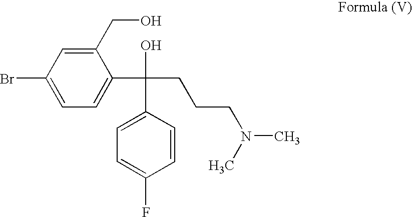

The fluoro-Grignard compound, III, is added to a cold solution of 5-bromopthalide (Formula II) slowly over 4-6 hours followed by the addition of the amino-Grignard compound, IV, at −5 to −6° C. The resultant mixture is stirred at −5 to −10° C. for 2 hours and additionally for 3 hours at room temperature. The molar excess of magnesium halides of 4-fluorobromo benzene (III) and N,N-dimethylaminopropyl chloride (IV) used in this reaction stage is typically from about 1 to about 2 fold, preferably is about 1.5 fold, relative to the 5-bromophthalide (II). Tetrahydrofuran is used in the present reaction at about 1 to 5 times; more particularly 1 to 2 times the amount of 5-bromophthalide, which provides optimum yield and acceptable purity of bromodiol (V).

The low temperature is employed in the present reaction to reduce the side products. After the desired time, the organic solvent used in the reaction is distilled under industrial vacuum between 55 to 65° C. Acetic acid is added to the residue to make it neutral to slightly basic. The residue from the reaction is extracted with ethyl acetate and basified to a pH between 8.0 to 9.0 using concentrate ammonia solution. Finally, ethyl acetate extracted bromodiol is crystallized by cooling the solution at 0° C. The product is filtered and dried in an oven at 60° C. for about 4 hours. HPLC Purity=99.20%, Melting Point=155 to 160° C., Moisture Content=2.30%.

The bromodiol prepared in the present study is a crystalline solid, which has not been well described in the literature. Preparation of bromodiol in solid crystalline form increases its purity and this is important for obtaining high purity product.

### (b) Cyclization of Bromodiol (Bromocitalopram)

The bromodiol (Formula V) is cyclized under very mild conditions using methane-sulphonyl chloride and triethylamine to form bromocitalopram (Formula VI): 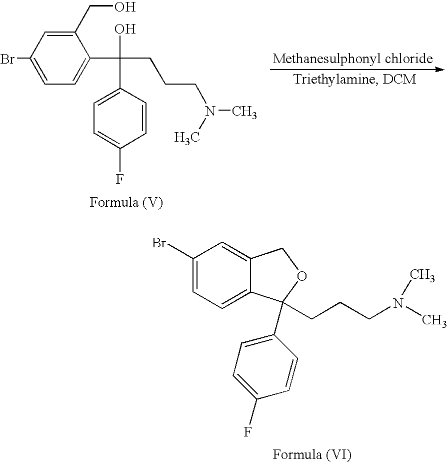
The bromodiol is charged in a reaction vessel in an aliphatic halide solvent, such as, for example, dichloromethane, followed by the addition of triethylamine. The reaction mixture is cooled to −5° C. and a solution of methanesulfonyl chloride in dichloromethane is added. The reaction mixture is warmed to room temperature and stirred for about 1-2 hours until the reactant (Bromodiol) disappears. After usual work-up, crude bromocitalopram is dissolved in petroleum ether and filtered to remove insoluble impurities. This process provides more than 97% HPLC pure bromocitalopram.

### (c) Preparation of Citalopram

Citalopram (Formula I) is prepared using an exchange reaction starting with (4′-fluorophenyl)-1-(3-dimethylaminopropyl)-5-bromophthalan (Formula VI) and copper cyanide (cyanide salt) in a polar solvent:

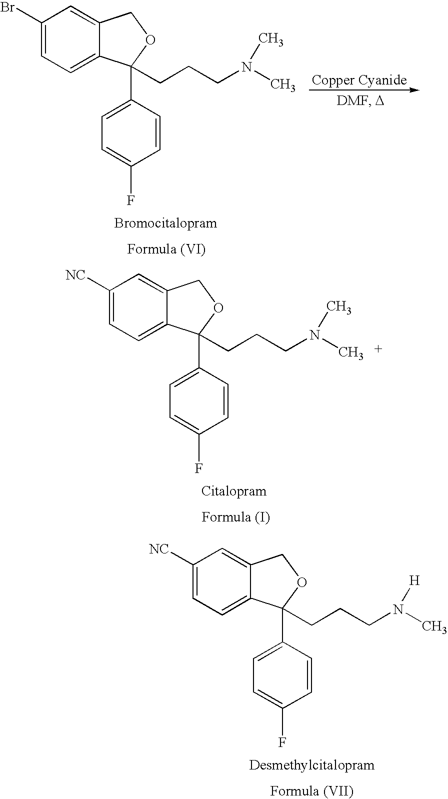

Suitable polar solvents for this reaction include dimethylformamide. The molar ratio of cyanide salt is from about 1 to 5, and more preferably from about 2 to 3 times the amount of bromocitalopram. Copper cyanide reacts with bromocitalopram at high temperature, preferably about 160° C. The molar ratio of copper cyanide is important in this reaction. A molar ratio of copper cyanide to bromocitalopram of from about 2:1 to about 3:1 is preferred. More preferably the ratio of copper cyanide to bromocitalopram is of about 2.5:1. This ratio helps to provide the maximum conversion of the bromo group into the cyano group.

The exchange reaction is continued until the formation of citalopram is complete, as unreacted bromocitalopram is difficult to remove from the final product. A simplified work-up procedure of this reaction is as follows: the reaction mixture is poured into a mixture of aqueous ethylenediamine and chloroform, stirred and filtered to remove metallic and other solid impurities. Citalopram is extracted with chloroform. This simplified work-up procedure avoids use of sodium cyanide for the removal of copper impurities as disclosed in U.S. Pat. No. 4,136,193. The crude citalopram obtained after extraction contains approximately 5-10% desmethylcitalopram.

Due to the structural similarity between desmethylcitalopram and citalopram, it is very difficult to remove desmethylcitalopram from citalopram by simple re-crystallization operation. The WO 2001/045483 document discloses a method for the purification of citalopram, especially removal of desmethylcitalopram. This method is such that it only removes desmethylcitalopram.

However, in the present invention desmethylcitalopram is reconverted into citalopram. This process removes the impurity as well as increases the yield of citalopram. The crude citalopram is isolated by chloroform extraction from the reaction mixture and purified by treatment with acetic acid and ammonia.

The crude chloroform solution containing citalopram and desmethylcitalopram is treated with formic acid and formaldehyde as shown below:

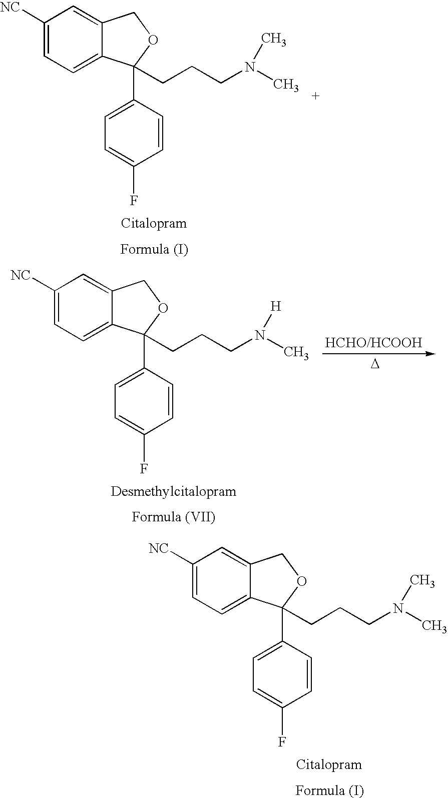

### (d) Preparation of Citalopram hydrobromide

Citalopram synthesized above is treated with 48% aqueous hydrobromic acid in a mixture of water and isopropyl alcohol at room temperature to afford crude crystalline citalopram bromide salt:

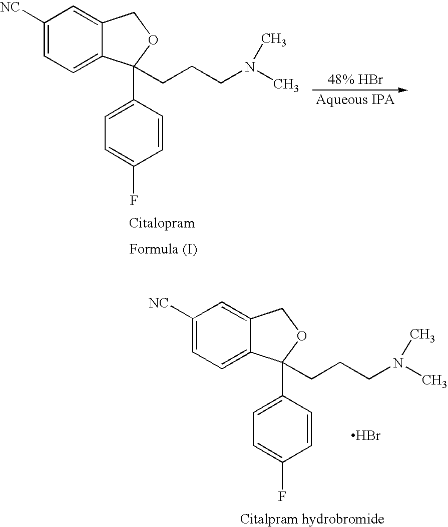

The resultant crystalline citalopram hydrobromide is stirred for 8 to 10 hours. The temperature is controlled between 30 to 35° C. to provide conditions for the formation of high quality hydrobromide. Increase of temperature during hydrobromide formation can lead to degradation of citalopram. Finally, the citalopram hydrobromide obtained is re-crystallized from aqueous isopropyl alcohol to provide highly pure citalopram hydrobromide.

The following examples illustrate the invention, but is not limiting thereof.

### EXAMPLE 1

### (4-Bromo-2-hydroxymethyl)phenyl-(4-fluorophenyl)-3-(dimethylaminopropyl)methanol, (Bromodiol)

N,N-Dimethylaminopropyl chloride hydrochloride, 1.0 L, (as a 60% aqueous solution) is cooled to 0° C. and 0.40 kg 50% caustic lye is added to it under constant stirring. The solution is allowed to warm to room temperature and the layers are separated. The upper organic layer is dried over sodium hydroxide flakes and distilled under vacuum, fractions boiling between 50 to 55° C. at 60 mm of Hg is collected to obtain 425 gm of pure dry N,N-dimethyl-aminopropyl chloride.

A first 10 L three-necked, round bottomed flask equipped with a stirrer, thermowell, and nitrogen inlet, is charged with magnesium turnings (104 gm), 200 ml tetrahydrofuran and a crystal of iodine. A solution of 4-fluorobromobenzene (0.624 Kg) in tetrahydrofuran (800 ml) is added to the first flask, slowly over 2 hours to obtain the fluoro-Grignard reagent, 4-fluorophenylmagnesium bromide.

A second similarly equipped 10 L three-necked, round bottomed flask, is charged with magnesium turnings (104 gm), 200 ml tetrahydrofuran and a crystal of iodine. A solution of N,N-dimethylaminopropyl chloride (410 gm) in 500 ml tetrahydrofuran is added to the second flask over 2 hours to obtain the amino-Grignard reagent, N,N-dimethylamino-propylmagnesium chloride.

A 20 L round bottom flask is charged 5-bromophthalide (532 gm), tetrahydrofuran (800 ml) and cooled to −5 to −10° C. The 4-fluorophenylmagnesium bromide/tetrahydrofuran solution is added slowly over 4 to 6 hours to the 5-bromophthalide solution. After addition of the fluoro-Grignard solution, the N,N-dimethylamino-propylmagnesium chloride/tetrahydrofuran solution is added over 4 to 6 hours at −5 to −6° C. The reaction mixture is stirred at −5 to −10° C. for an additional 2 hours and allowed to warm to room temperature, and stirred for an additional 3 hours. After the reaction is completed, it is cooled to 0 to −5° C., water (10 L) is added, and distilled under industrial vacuum at 60 to 65° C. to recover tetrahydrofuran. Acetic acid (500 ml) is added to obtain a pH of about 7.0 to 8.0. The reaction products are extracted with ethyl acetate (3×2.5 L). The combined ethyl acetate layers are re-extracted with 20% acetic acid in water (3×2.5 L). Acetic acid extract is basified with concentrated ammonia (1.90 L) to pH 8.0 to 9.0. The bromodiol is extracted with fresh ethyl acetate (3×3.0 L). The combined ethyl acetate extract is cooled to 0° C. and stirred for 3 hours, which results in complete crystallization of bromodiol. The product is filtered and dried in oven at 60° C. for 4 hours. Yield of bromodiol=556 gm, HPLC Purity=99.20%, Melting Point=155 to 160° C., Moisture Content=2.30%.

### EXAMPLE 2

### 1-(4′-Fluorophenyl)-1-(3-dimethylaminopropyl)-5-bromophthalan (Bromocitalopram)

A 20 L round bottomed flask equipped with a stirrer, thermowell, and addition funnel, is charged with bromodiol (550 gm), dichloromethane (9.70 L), and triethylamine (571 ml). The resultant mixture is cooled to −5° C. and a solution of methanesulfonyl chloride (139.5 ml) in dichloromethane (550 ml) is added to it over 1 hour. The reaction mixture is warmed to room temperature and stirred for about 1 to 2 hours until the starting material disappears. The reaction mixture is washed with aqueous sodium hydroxide solution (0.1 N, 2×5.50 L) followed by water (2×5.0 L), dried over sodium sulfate and concentrated to obtain the crude bromocitalopram as a syrup. Yield of crude bromocitalopram=390 gm. HPLC Purity=90%.

Crude bromocitalopram is refluxed with petroleum ether (60-80° C., 8.0 L). The solution is filtered while hot to remove insoluble impurities. The product is concentrated to provide purified bromocitalopram. Yield of Bromocitalopram=350 gm, HPLC Purity=97.6%.

### EXAMPLE 3

### 1-(4′-Fluorophenyl)-1-(3-dimethylaminopropyl)-5-phthalanecarbonitrile (Citalopram)

A 2.0 L round bottomed flask equipped with a stirrer, nitrogen inlet, thermowell and reflux condenser, is charged with dimethylformamide (240 ml) and copper cyanide (207 gm) and heated under stirring at 150° C. (gentle reflux). To this mixture, a solution of bromocitalopram (350 gm) in dimethylformamide (70 ml) is added. About 70 ml of dimethylformamide is distilled out at atmospheric pressure from the reaction mixture. This raises the reaction temperature to 163-165° C. and is maintained under stirring for 6 to 8 hours. The reaction mixture is allowed to cool to 60° C. and is added into a mixture of ethylenediamine (380 ml) and water (1.80 L) under nitrogen stirring. The reaction mixture is allowed to cool to 40° C. and chloroform (1.40 L) is added. The mixture is stirred for half an hour and filtered to remove metallic impurities. The filtrate is allowed to separate in two layers. The lower, organic layer is separated, followed by re-extraction of aqueous layer with chloroform (2×0.80 L). The combined chloroform extracts are washed with water (0.50 L).

Formic acid (0.31 L) and formaldehyde (0.29 L) are added to the chloroform layer and heated at reflux for 8 hours. The reaction mixture is cooled to room temperature and basified to pH 8.0 to 9.0 using ammonia solution. The chloroform layer is separated, washed with water (0.80 L), dried over sodium sulfate and concentrated to thick residue (308 gm). The residue is dissolved in toluene (1.80 L). The toluene solution is extracted using 20% aqueous acetic acid (3×1.20 L). The combined aqueous extracts are basified to pH 8.0 to 9.0 using a sodium hydroxide solution (0.75 L). The citalopram obtained is extracted using isopropyl ether (3×1.20 L), the combined extracts are washed with water, dried over sodium sulfate and concentrated under industrial vacuum to obtain Citalopram free base as a thick oily residue. Yield of Citalopram=180 gm, HPLC Purity=90 to 94%.

### EXAMPLE 4

### 1-(4′-Fluorophenyl)-1-(3-dimethylaminopropyl)-5-phthalanecarbonitrile hydrobromide (Citalopram hydrobromide)

A 1.0 L round bottomed flask equipped with a stirrer, and a thermowell, is charged with a mixture of water (160 ml) and isopropyl alcohol (40 ml). Citalopram free base (180 gm) is added and the mixture is stirred at room temperature followed by addition of 48% hydrobromic acid (35 ml) (forms a clear solution). The reaction mixture is cooled to 0° C. to obtain crystalline citalopram hydrobromide. Stirring is continued at 0° C. for 8 to 10 hours and the solid product filtered to obtain wet citalopram hydrobromide. Yield of wet Citalopram Hydrobromide=150 gm.

The wet citalopram hydrobromide is dissolved in a mixture of water (160 ml) and isopropyl alcohol (40 ml) at 35 to 40° C., charcoalized using 5 gm charcoal and filtered through a hyflow bed. The filtrate is allowed to stir at 0° C. for about 4 to 5 hours to obtain crystalline citalopram hydrobromide, and filtered. The crystalline solid is dried under vacuum in an oven at 60° C. for 4 to 5 hours to obtain re-crystallized citalopram hydrobromide. Yield of Citalopram Hydrobromide=85 gm, HPLC Purity=99.60%. The re-crystallization procedure is repeated till a desired purity level of citalopram is obtained.

All patents, patent applications, and literature cited in the specification are hereby incorporated by reference in their entirety. In the case of any inconsistencies, the present disclosure, including any definitions therein will prevail. The invention has been described with reference to various specific and preferred embodiments and techniques. However, it should be understood that many variations and modifications may be made while remaining within the spirit and scope of the invention.

What is claimed is:

1. A process for manufacturing a compound of Formula (1) 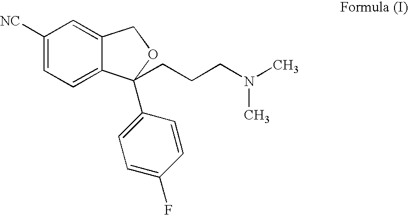
and its salts, said method comprising:
(a) subject a compound of the (VI) 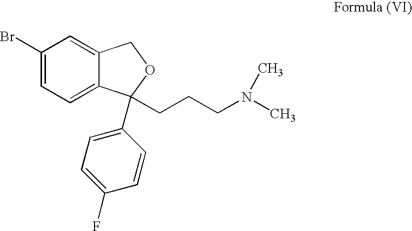
to cyanide exchange reaction until complete substitution of the bromo group;
(b) optionally purify the crude citalopram;
(c) heat citalopram from step (a) or (b) with a mixture of aldehyde and acid to convert desmethylcitalopram into citalopram base; and
contacting pure citalopram base with aqueous hydrogen halide, and isolating the pure product.

2. The process of claim 1, wherein step (a) is carried out in an organic solvent.

3. The process of claim 2, wherein the organic solvent is N,N-dimethylformamide.

4. The process of claim 1, wherein 5-bromocitalopram is added to a pre heated copper cyanide in N,N-Dimethylformamide.

5. The process of claim 4, wherein 5-bromocitalopram in N,N-dimethylformamide solution is added.

6. The process of claim 1, wherein the reaction temperature is from about 150 to 170° C.

7. The process of claim 1, wherein the completed reaction in step (b) is cooled to about 60° C. and poured into a basic solution.

8. The process of claim 7, wherein the basic solution comprises ethylenediamine and water in the ratio between about 1.0:1.0 to about 1.0:5.0.

9. The process of claim 1, wherein the aliphatic halide solvent in step (b), is added to the reaction mixture.

10. The process of claim 9, wherein said aliphatic halide is chloroform.

11. The process of claim 1, wherein said acid in step (c) is an aliphatic acid.

12. The process of claim 11, wherein said aliphatic acid is formic acid.

13. The process of claim 1, wherein said aldehyde in step (c) is an aliphatic aldehyde.

14. The process of claim 13, wherein said aldehyde is formaldehyde.

15. The process of claim 1, wherein molar ratio of formic acid and formaldehyde, in step (c), is between about 0.50 to about 3.0.

16. The process of claim 1, wherein reaction mixture in step 1 (c) is heated to reflux temperature.

17. The process of claim 1, wherein the heating time of reaction mixture, in step (c), is between about 1 to about 20 hours.

18. The process of claim 17, wherein reaction mixture is cooled to room temperature and basified.

19. The process of claim 18, wherein said basic source is concentrated ammonia.

20. The process of claim 19, wherein pH of the said crude solution is in between about 7.0 to about 10.0.

21. The process of claim 20, wherein chloroform layer is separated, washed with water and concentrated to get crude citalopram.

22. The process of claim 21, wherein said product is dissolved in aromatic hydrocarbon.

23. The process of claim 22, wherein said aromatic hydrocarbon is toluene.

24. The process of claim 23, wherein said solution is extracted with aqueous acid.

25. The process of claim 24, wherein said acid is 20% acetic acid in water.

26. The process of claim 25, wherein aqueous layer is basified by a metallic hydroxide.

27. The process of claim 26, wherein pH of the said solution is between about 6.5 to about 11.0.

28. The process of converting, desmethylcitalopram having the formula 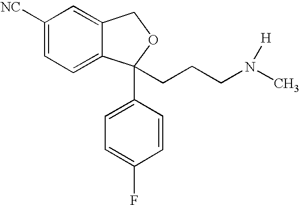
to citalopram.

29. The process of claim 1, wherein citalopram is converted to citalopram hydrobromide in step (c) by using aqueous hydrogen bromide.

30. The process of claim 29, wherein reaction is carried out in aqueous alcohol.

31. The process of claim 30, wherein said alcohol is isopropanol.

32. A crystalline compound having the Formula 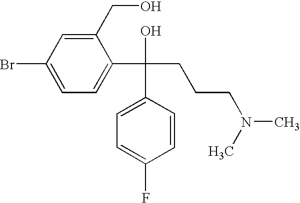
in substantially pure form.

33. A process for cyclization of the compound having formula 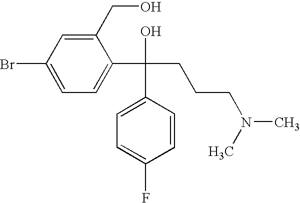
into a compound having formula 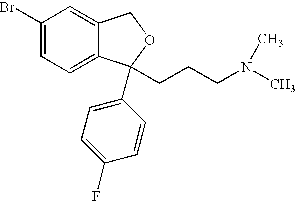
in an organic solvent in the presence of a sulphonyl halide and a base.

34. The process of claim 33, wherein said organic solvent is aliphatic halide.

35. The process of claim 34, wherein said halide is sulphonyl halide.

36. The process of claim 35, wherein said sulphonyl halide is methanesulphonyl chloride.

37. The process of claim 33, wherein said base is aliphatic amine.

38. The process of claim 37, wherein more particularly aliphatic amine is triethylamine.

39. The process of claim 33, wherein solution is stirred at room temperature for about 1 to about 2 hours.

40. The process of claim 1, wherein less than 0.06% desmethylcitalopram remains in step (C) after acid-aldehyde treatment.
